(19) United States
(12) Patent Application Publication
(10) Pub. No.: US 2024/0108855 A1
(43) Pub. Date: Apr. 4, 2024

(54) DEVICES, SYSTEMS, AND METHODS FOR PERCUTANEOUS- MEDIATED FLUID REMOVAL

(71) Applicant: German Antonio ROSERO, Bogotá (CO)

(72) Inventor: German Antonio ROSERO, Bogotá (CO)

(21) Appl. No.: 18/239,016

(22) Filed: Aug. 28, 2023

Related U.S. Application Data
(60) Provisional application No. 63/412,578, filed on Oct. 3, 2022.

(51) Int. Cl. A61M 25/00

(52) U.S. Cl. CPC A61M 25/0082; A61M 25/0021

(57) ABSTRACT

The present disclosure is directed to devices, systems, and methods for percutaneous applications and/or uses of negative pressure therapy (NPT). Specifically, a novel catheter (e.g., a percutaneous drainage catheter) and a suction apparatus and/or system (e.g., a vacuum system) is disclosed. The novel catheter may include one or more sponges for absorbing one or more fluids from the body cavity of a subject or patient. Further, the suction apparatus and/or system may be configured to apply NPT. In at least one embodiment, negative pressure is applied through the catheter and/or the one or more sponges, thereby allowing fluids (e.g., abscesses, exudate, harmful material, infections material and/or infections agents, etc.) to be collected, drained, and/or otherwise removed from the body cavity of a subject or patient.

### STATEMENT REGARDING FEDERALLY SPONSORED RESEARCH

This invention was made without government support or grants awarded by the National Institutes of Health. The government has no rights in the invention.

### FIELD OF THE DISCLOSURE

The present disclosure relates to devices, systems, and methods for removal of one or more fluids, including, for instance, abscesses, exudate, and/or other harmful material (e.g., infections material and/or infections agents), from a body cavity of a subject or patient. In particular, various embodiments of the disclosure relate to percutaneous-mediated removal of such fluids. Specifically disclosed herein is a novel catheter (e.g., a percutaneous drainage catheter) and a suction apparatus and/or system (e.g., a vacuum system) that may be configured to apply negative pressure therapy (NPT). The catheter may, in at least one embodiment, comprise one or more sponges attached thereto, such that the suction apparatus and/or system is capable of transferring negative pressure through the catheter and/or through the one or more sponges, thereby allowing fluids to be removed from the body cavity of the subject or patient.

### BACKGROUND

Various devices are used in medical procedures to remove fluids from, and/or deliver fluids into, a body cavity of a patient. Such devices include catheters, which can be inserted into a body cavity, often through a narrow opening or incision. Catheters can be used, for example, to drain exudate from the abdominal cavity.

However, known catheters suffer from various deficiencies in removing fluid from a patient's body cavity. For instance, catheters must often be introduced precisely into an area and/or cavity for which fluid must be removed. This can require the use of long, thin needles and time-consuming procedures on the patient, including the use of image guides and wire sheets to introduce the catheter and/or needle into the patient. Moreover, catheters may not remove all of the fluid that needs to be removed. Additional harmful material (e.g., abscesses, infection-causing particles, bacteria, viruses, etc.) may be left inside the patient's body cavity, requiring multiple procedures to remove and/or drain all of the fluid or material. Moreover, any leftover harmful material can inhibit and/or prevent wound healing, infection clearance, recovery from surgery and/or operations, and the like.

In view of the foregoing, there is a significant need for devices, systems, and methods that can be used to efficiently collect and/or drain fluid from a body cavity of a subject, and that are affordable, readily accessible, and easy to use for both a medical professional and a subject. In particular, there is a need for devices, systems, and methods that provide for percutaneous applications of fluid drainage from a body cavity, thereby minimizing surgical incisions for the subject and avoiding opening and/or exposure of the entire body cavity.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards devices, systems, and methods for the removal of fluids (e.g., abscesses, exudate, and/or harmful material, including, for instance, infections material and/or infections agents) from a subject or patient, and, in particular, from one or more body cavities of the patient. Such devices, systems, and methods can be achieved using percutaneous insertion of fluid removal devices and/or systems described herein. Specifically described herein are fluid removal devices, systems, and methods that utilize one or more catheters physically connected and/or attached to one or more sponges, which can be used in conjunction with one or more suction, vacuum and/or negative pressure devices and/or systems.

In at least one embodiment, a fluid removal device comprises a catheter (e.g., a percutaneous catheter) and one or more sponges that may be physically connected and/or attached to the catheter. In at least one example, the catheter comprises a plurality of holes disposed at a proximal end of the catheter near the catheter tip. The catheter may also have an interior lumen in the axial direction, spanning from the proximal end to the distal end. In at least an additional example, the one or more sponges can be fitted over an outside circumference of the catheter at, for instance, the proximal end of the catheter. The sponge can therefore be of a cylindrical, or substantially cylindrical, shape with a hollow central portion such that the proximal end of the catheter can be inserted within the central portion. In at least a further example, the one or more sponges are microporous such that fluid (e.g., any fluid to be removed from the body cavity of a patient) can enter and/or pass through the one or more sponges. Accordingly, the one or more sponges can be absorbable and/or absorbent. Further, the one or more sponges can be composed of one or more suitable materials, including, for instance, polyurethane, gel/gelatin, and the like.

In at least an additional embodiment, a fluid removal device comprises a curved catheter (e.g., a percutaneous catheter) and one or more sponges that may be physically connected and/or attached to the catheter. In at least one example, the catheter may be curved (e.g., at a proximal end) before or after entering the patient in order to, for instance, provide better access to fluid in the body cavity of the patient, thereby enabling better and/or more efficient removal of such fluid. The curved catheter may comprise a plurality of holes disposed at the proximal end of the catheter near the catheter tip. The catheter may also have an interior lumen in the axial direction, spanning from the proximal end to the distal end. In at least an additional example, the one or more sponges can be fitted over an outside circumference of the catheter at, for instance, the proximal end of the catheter. The sponge can therefore be of a cylindrical, or substantially cylindrical, shape with a hollow central portion such that the proximal end of the catheter can be inserted within the central portion. Additionally, the one or more sponges are sufficiently flexible such that they accommodate and/or adapt to the curved portion of the catheter. In at least a further example, the one or more sponges are microporous such that fluid (e.g., any fluid to be removed from the body cavity of a patient) can enter and/or pass through the one or more sponges. Accordingly, the one or more sponges can be absorbable and/or absorbent. Further, the one or more sponges can be composed of one or more suitable materials, including, for instance, polyurethane, gel/gelatin, and the like.

In at least a further embodiment, a fluid removal device comprises a catheter (e.g., a percutaneous catheter), one or more sponges that may be physically connected and/or attached to the catheter, and one or more cover layers that may be physically connected and/or attached to the one or more sponges. In at least one example, the catheter may comprise a plurality of holes disposed at the proximal end of the catheter near the catheter tip. The catheter may also have an interior lumen in the axial direction, spanning from the proximal end to the distal end. In at least an additional example, the one or more sponges can be fitted over an outside circumference of the catheter at, for instance, the proximal end of the catheter. The sponge can therefore be of a cylindrical, or substantially cylindrical, shape with a hollow central portion such that the proximal end of the catheter can be inserted within the central portion. In at least a further example, the one or more sponges are microporous such that fluid (e.g., any fluid to be removed from the body cavity of a patient) can enter and/or pass through the one or more sponges. Accordingly, the one or more sponges can be absorbable and/or absorbent. Further, the one or more sponges can be composed of one or more suitable materials, including, for instance, polyurethane, gel/gelatin, and the like.

In at least one example, the cover layer may be composed of one or more suitable materials (e.g., cellulose). The cellulose may, in at least one example, be an absorbable and/or absorbent cellulose. In at least an additional example, the cover layer comprises one or more releasable portions that may allow the cover layer to be released from the sponge. Such release can happen either before or after insertion of the fluid removal device into the body cavity of the patient. The one or more releasable portions may, in at least a further example, comprise a pre-cut portion or line, removal of which causes the cover layer to detach from the sponge. For instance, the releasable portion may be pre-cut such that exertion of force (e.g., on a suture physically connected and/or attached to the releasable portion) on such portion causes the cover layer to detach from the sponge. The cover layer can then be removed from the body cavity of the patient (e.g., by a medical professional or clinician treating the patient).

In at least a further example, the cover layer constricts and/or restricts the diameter and/or size of the sponge, which may be necessary for insertion of the fluid removal device into the body cavity of the patient (e.g., through one or more incisions in the skin and/or tissues of the patient). Removal of the cover layer (e.g., via removal of the one or more releasable portions) can occur after the fluid removal device has been sufficiently inserted into the body cavity of the patient. Such removal may cause the sponge to expand to its normal, uncompressed diameter and/or size. Accordingly, various different sizes of sponges can be used in the fluid removal devices described herein, depending on the area of the body cavity surface to be drained.

In at least a further embodiment, a fluid removal device is disclosed herein that can be used in conjunction with a suction, vacuum and/or negative pressure device and/or system (e.g., a negative pressure (NPT) device and/or system). Such a suction, vacuum and/or negative pressure device and/or system can provide negative pressure and/or sub-atmospheric pressure to assist in extracting fluid from the fluid removal device. In at least one example, any fluid removal device disclosed herein can be used in conjunction with a suction, vacuum and/or negative pressure device and/or system.

In at least a further embodiment, fluid can then be extracted from the body cavity of a patient into, and through, the sponge and into the catheter of a fluid removal device through the plurality of holes disposed on the catheter. The sponge can therefore provide an absorbable and/or absorbent surface that can draw the fluid from the body cavity through the plurality of holes into the catheter. The fluid can then pass through the catheter from the proximal end through the distal end and out of the catheter. This brings the fluid out of the catheter; the fluid can then be collected using one or more collection devices (e.g., collection bags). Removal of such fluid, as mentioned above herein, can be achieved, either partially or wholly, through the use of a suction, vacuum and/or negative pressure device and/or system (e.g., a negative pressure therapy (NPT) device and/or system). Such a suction, vacuum and/or negative pressure device and/or system can be physically connected and/or attached to any of the fluid removal devices herein (e.g., through the distal end of a catheter). Activation of the negative pressure device and/or system can cause negative and/or sub-atmospheric pressure to be applied to, and/or exerted on, the fluid removal device and, in particular, the catheter and the sponge. This can cause the fluid to be extracted from the patient's body cavity into the sponge and then into the catheter. In at least one example, the applied negative and/or sub-atmospheric pressure is in the range of −40 mM Hg to −200 mM Hg; that is, 40 mM Hg below atmospheric pressure to 200 mM Hg below atmospheric pressure, or more preferably −80 mM Hg to −150 mM Hg, or still more preferably −100 mM Hg to −150 mM Hg.

In at least a further embodiment, a fluid removal system is disclosed herein. Such a fluid removal system incorporates suction, vacuum, and/or negative pressure. In at least one example, the fluid removal system comprises a catheter (e.g., any of the catheters described herein) and a sponge (e.g., any of the sponges described herein). The distal end of the catheter is physically connected and/or attached to a suction, vacuum, and/or negative pressure device. Such a negative pressure device and/or system may be, for instance, a NPT device and/or system. Negative and/or sub-atmospheric pressure can be applied to, and/or exerted on, the catheter and the sponge. This results in fluid being drawn into the sponge. Such movement removes the fluid from the body cavity of the patient. The fluid is absorbed by the sponge and the fluid then passes into the catheter and moves towards the distal end of such catheter. Since the distal end is physically connected and/or attached to the negative pressure device, the fluid is drawn from the distal end of the catheter into the negative pressure device. The fluid can then be collected (e.g., in one or more collection devices and/or collection bags) and/or otherwise disposed of.

In at least a further embodiment, methods for applying one or more fluid removal devices and/or systems are disclosed.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 depicts a conventional catheter known in the art.

FIG. 2 depicts a perspective view of a fluid removal device that can be used in conjunction with a suction, vacuum and/or negative pressure device and/or system, according to at least one embodiment of the disclosure.

FIG. 3 depicts a perspective view of a fluid removal device that can be used in conjunction with a suction, vacuum and/or negative pressure device and/or system, in a non-limiting position of use, according to at least one embodiment of the disclosure.

FIGS. 4A-4B depict a fluid removal device that can be used in conjunction with a suction, vacuum and/or negative pressure device and/or system, in both a perspective view (FIG. 4A) and a view in the axial direction from the proximal end (FIG. 4B), according to at least one embodiment of the disclosure.

FIGS. 5A-5B depicts perspective views of a fluid removal device in non-limiting positions of use in conjunction with conjunction with a suction, vacuum and/or negative pressure device and/or system, according to at least one embodiment of the disclosure.

FIG. 6 depicts a fluid removal system incorporating suction, vacuum, and/or negative pressure, according to at least one embodiment of the disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may affect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise, the terms “include,” “including,” and “or” should all be construed to be inclusive, unless such a construction is clearly prohibited from the context. The terms “comprising” or “including” are intended to include embodiments encompassed by the terms “consisting essentially of” and “consisting of” Similarly, the term “consisting essentially of” is intended to include embodiments encompassed by the term “consisting of” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

In describing embodiments of the disclosure, it will be understood that a number of techniques and steps are disclosed. Each of these has individual benefit(s) and each can also be used in conjunction with one or more, or in some cases all, of the other disclosed techniques. Accordingly, for the sake of clarity, the present disclosure shall refrain from repeating every possible combination of the individual steps in an unnecessary fashion.

In the present description, for purposes of explanation, numerous specific details are set forth in order to provide a thorough understanding of one or more of the embodiments. It will be evident, however, to one skilled in the art that one or more embodiments of the disclosure may be practiced without these specific details.

The present disclosure is to be considered as providing non-limiting embodiments and/or exemplifications, and is not intended to limit the invention to any one or more specific embodiments and/or exemplifications illustrated and/or described herein.

### Definitions

The following is a non-exhaustive and non-limiting list of terms used herein and their respective definitions.

The terms “agent” or “active agent,” which are used interchangeably herein, refer to a physiologically or pharmacologically active substance that acts locally and/or systemically in a subject's body. An “agent” or “active agent” is a compound or substance that is administered to an individual for the treatment (e.g., therapeutic agent, cancer therapeutic agent, and the like), prevention (e.g., prophylactic agent), or diagnosis (e.g., diagnostic agent) of a disease or disorder. Such agents may also include therapeutics that prevent or alleviate symptoms, such as, for instance, symptoms associated with one or more disorders and/or conditions.

The term “administering” or “administration” refers to providing or giving a subject one or more agents, formulations, and/or medical devices and/or medical therapies, either alone or in conjunction with any other agent, formulation, and/or medical device and/or medical therapy, by any effective route. Exemplary routes of administration include, but are not limited to, percutaneous, injection (such as, e.g., subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation.

The term “axial direction,” at least as used herein, refers to the longitudinal direction of an object, item, or device (e.g., a catheter) from the proximal end of the object, item, or device to the distal end of the object, item, or device, or vice versa.

The term “catheter” refers to a specific tool or device used in medicine (including, but not limited to, medical procedures and/or surgeries), and, in particular, a flexible tube inserted through one or more openings into a body cavity of a subject. Exemplary body cavities include, but are not limited to, the bladder, the abdominal cavity, the chest cavity, one or more canals, one or more vessels, one or more passageways, and the like. Catheters can be used for various non-limiting purposes, including, for instance, permitting injection into a body cavity, removing or withdrawing abscesses, exudate, and/or fluids from a body cavity, and/or keeping a body cavity open (e.g., for medical procedures and/or surgeries).

The terms “decrease,” “lower,” “lessen,” “reduce,” and “abate,” which are used interchangeably herein, refer generally to the ability of a compound, formulation, medical device, and/or medical therapy (including those disclosed herein) to produce, elicit, and/or cause a lesser physiological response (e.g., downstream effects) compared to the response caused by a respective control compound, formulation, medical device, and/or medical therapy. A “decrease” or “reduced” amount is typically a “statistically significant” amount, and may include a decrease that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7, 1.8, etc.).

The term “distal end,” at least as used herein, refers to an axial end of an object, item, or device (e.g., a catheter) that is closer to, and/or guided and/or held by, an individual operating the object, item, or device on a subject or patient. A non-limiting example of such an individual could be, for instance, a medical professional (e.g., doctor, nurse, surgeon, etc.).

The terms “enhance,” “induce,” “induction,” and “increase,” which are used interchangeably herein, refer generally to the ability of a compound, formulation, medical device, and/or medical therapy (including those disclosed herein) to produce, elicit, and/or cause a greater physiological response (e.g., downstream effects) compared to the response caused by a respective control compound, formulation, medical device, and/or medical therapy. An “enhanced” or “increased” amount is typically a “statistically significant” amount, and may include an increase that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7, 1.8, etc.).

The term “exudate” refers to a mass of cells and/or fluid that has emerged, and/or seeped out of, one or more portions of the body (e.g., one or more blood vessels, one or more organs, etc.). Accordingly, the term “exudate” encompasses cells and/or fluid emitted through one or more pores and/or wounds. As a non-limiting example, exudate can be emitted out of one or more organs into a body cavity (e.g., the abdominal cavity, the chest cavity, the bladder, etc.) of a subject. Exudate can occur as a result of various diseases and/or conditions (e.g., inflammation).

The terms “negative pressure therapy” or “NPT,” which are used interchangeably herein, refer generally to a medical procedure and/or medical therapy, and/or one or more medical devices or systems that perform such a procedure and/or therapy, that applies negative pressure or sub-atmospheric pressure to one or more portions of a subject. Negative pressure therapy can be used in various medical applications to remove abscesses, exudate, and/or fluids from one or more portions of a subject (e.g., one or more body cavities, including, but not limited to, the abdominal cavity, the chest cavity, the bladder, and the like). Accordingly, negative pressure can be used in, for instance, wound care treatment.

The term “percutaneous,” at least as used herein, is defined as being made, done, effected, occurring, and/or performed through the skin.

The term “proximal end,” at least as used herein, refers to an axial end of an object, item, or device (e.g., a catheter) that is closer to, and/or inserted into, a subject or patient.

The terms “subject,” “individual,” or “patient,” which are used interchangeably herein, refer to a vertebrate, such as a mammal (e.g., a human). Mammals include, but are not limited to, murines (e.g., mice), simians, humans, farm animals, sport animals, and pets. In at least one embodiment, the subject is a non-human mammal, such as a monkey or other non-human primate, mouse, rat, rabbit, guinea pig, pig, goat, sheep, dog, cat, horse, or cow. In at least one example, the subject can be treated using one or more agents, formulations, medical devices, medical therapies, and/or methods (e.g., including one or more medical devices and/or medical therapies) disclosed herein. In at least an additional example, the subject is a laboratory animal/organism, such as, for example, a mouse, rabbit, guinea pig, or rat. In at least a further example, a subject includes, for instance, farm animals, domestic animals and/or pets (e.g., cats, dogs). A “patient” can specifically refer to a subject that has been diagnosed with a particular disease, condition, and/or indication that can be treated with one or more agents, formulations, medical devices, medical therapies, and/or methods (e.g., including one or more medical devices and/or medical therapies, either alone or in conjunction with one or more other agents, formulations, medical devices, medical therapies, and/or methods) disclosed herein.

The term “transverse direction,” at least as used herein, refers to any direction that intersects the longitudinal axis of an object, item, or device (e.g., a catheter), at any angle.

The terms “treating,” “treatment,” and “therapy” refer, either individually or in any combination, to any success or indicia of success in the attenuation or amelioration of an injury, disease, symptom, disorder, pathology, and/or condition, and/or pathological condition, including any objective or subjective parameter such as, for instance, abatement, remission, diminishing of symptoms or making the condition more tolerable to the patient, slowing the rate of degeneration or decline, making the final point of degeneration less debilitating, improving a subject's physical or mental well-being, and/or prolonging the length of survival. Treatment does not necessarily indicate complete eradication or cure of the injury, disease, symptom, disorder, pathology, and/or condition, and/or pathological condition, or any associated symptom(s) thereof. The treatment may be assessed by one or more objective or subjective parameters, including, for example, the results of a physical examination, blood and other clinical tests (e.g., imaging), and the like. In at least one example, treatment with the disclosed one or more agents, formulations, medical devices, medical therapies, and/or methods (e.g., including one or more medical devices and/or medical therapies, either alone or in conjunction with one or more other agents, formulations, medical devices, medical therapies, and/or methods) results in a clinical improvement in one or more diseases and/or conditions in a subject.

Further, unless otherwise noted, technical terms are generally used according to conventional usage. Aspects of the disclosed methods employ, unless indicated specifically to the contrary, conventional methods of chemistry, biochemistry, organic chemistry, molecular biology, microbiology, cell biology, and/or surgery, many of which are described below solely for the purpose of illustration. Such techniques are explained fully in technical literature sources. General definitions of common terms in the aforementioned fields may be found in references known to one of ordinary skill in the art. Indeed, unless otherwise defined, all terms (including, but not limited to, technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Catheters

Various different types of medical devices can be used to remove fluids from, and/or deliver fluids into, one or more body cavities of a patient. For instance, catheters can be used to access one or more body cavities of a patient (e.g., abdominal cavity, chest cavity, bladder, etc.). Accordingly, catheters can be used to drain fluids from such body cavities. As an example, if a passage between a patient's kidney and bladder is blocked, a physical or clinician can insert a catheter into the patient's kidney to drain urine. Such catheters may therefore be connected to one or more drainage bags or similar apparatuses and/or devices to collect the fluid removed from the patient. Catheters may also be used to deliver fluids (e.g., medication, chemotherapy drugs) into the patient. Catheters may further be used as part of various diagnostic and/or therapeutic procedures. For example, they can be inserted into the vascular system of a patient to assist in the pumping and/or circulation of blood, for angioplasties, for electrophysiology applications, for blood pressure monitoring, for blood pressure sampling, and the like. As a still further use, catheters may be left in place as part of various medical procedures (e.g., for blood transfusions, for dialysis, etc.).

Insertion of a catheter may involve the use of one or more needles, image guides, guidewires, and/or wire sheets in order to insert the catheter efficiently and accurately. Depending on the specific medical application, additional medical devices (e.g., cannulae, imaging guidance devices such as ultrasound imagers, dilating devices, protective guards, additional intravenous (IV) and/or metal catheters, and the like) may be needed to introduce and/or use a catheter appropriately.

Percutaneous Catheters

Percutaneous catheters are one type of catheter. Such percutaneous catheters can be inserted through a patient's skin to access one or more body cavities. Insertion can be achieved using, for instance, one or more incisions into the patient's skin.

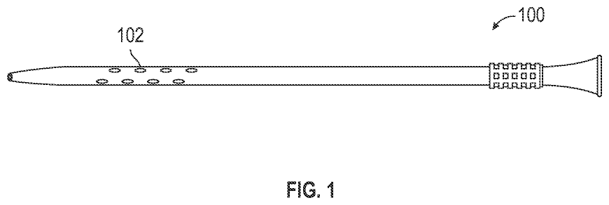

An example of a known type of percutaneous catheter, specifically a pig tail percutaneous catheter, is shown in FIG. 1. The catheter 100 contains a plurality of holes 102 for fluid to pass through from a portion of the human body (e.g., a patient's body cavity) into the catheter, or vice versa. As mentioned above herein, such fluids can be, for instance, exudate, abscesses, and/or other harmful material (e.g., infections material and/or infectious agents).

Although such known types of percutaneous catheters can be used for removing and/or draining fluid (e.g., abscesses, exudate, debris, infections material (including infections agents such as bacteria, viruses, and fungi), and the like) from a patient's body cavity, such use is subject to a variety of potential complications. For instance, precise insertion and/or placement of the percutaneous catheter may be required in order to remove the fluid. Moreover, even precise insertion and/or placement of the catheter may not remove all of the fluid. Incomplete removal of harmful material (e.g., exudate) may prevent and/or inhibit wound healing and/or infection clearance. Such incomplete removal may also result in the patient being required to undergo multiple catheter insertion procedures, leading to further discomfort for the patient and further risks of infection and/or inflammation.

Percutaneous-Mediated Fluid Removal

At least one embodiment of the present disclosure relate to devices, systems, and methods for percutaneous-mediated removal of fluids (e.g., abscesses, exudate, and/or harmful material, including, for instance, infections material and/or infections agents) from a body cavity of a subject or patient. Such percutaneous-mediated removal may include, in at least one embodiment, a fluid removal device comprising a percutaneous catheter and one or more sponges. In at least an additional embodiment, the fluid removal device can be used in conjunction with a suction, vacuum and/or negative pressure device and/or system. In at least a further embodiment, a fluid removal system is disclosed that comprises a fluid removal device and one or more suction, vacuum and/or negative pressure devices and/or systems. Methods for using a fluid removal device and a fluid removal system are also disclosed herein.

The aforementioned suction, vacuum and/or negative pressure devices and/or systems may include, for instance, negative pressure therapy (NPT) devices and/or systems; that is, devices and/or systems that can be used for NPT and/or exert negative pressure and/or sub-atmospheric pressure (e.g., through a pump, such as, for instance, an electronically-controlled pump). NPT is known by various alternate names, including, for instance, “negative pressure wound therapy” or “NPWT,” “vacuum-assisted closure” or “VAC,” “reduced pressure therapy,” “vacuum therapy,” “topical negative pressure,” and the like. NPT can be applied to open wounds and/or used for wound care treatment to help improve wound healing associated with, e.g., infections, trauma, and the like. Without wishing to be bound by theory, NPT can improve wound healing through various mechanisms, such as, for example, macroscopic and/or microscopic deformation of tissues, leading to increased blood flow and/or stimulation, improved changes to perfusion and/or blood flow regulation, control of exudate, and/or clearance of bacteria.

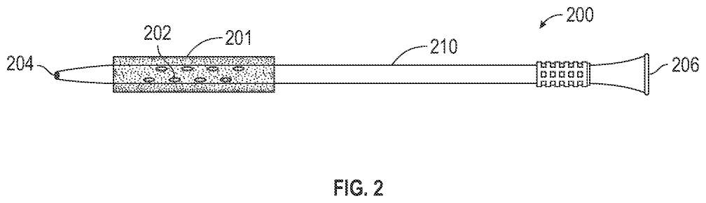

Turning now to FIG. 2, a fluid removal device 200 is shown according to at least one embodiment of the disclosure. The device 200 is shown in a perspective view and comprises a percutaneous catheter 210 and a sponge 201. The catheter 210 comprises a proximal end with a tip 204 and a distal end 206. A plurality of holes 202 are disposed at the proximal end near the tip 204. Further, the catheter 210 has an interior lumen in the axial direction, spanning from the proximal end to the distal end. The catheter may, in at least one example, have a hollow interior.

The sponge 201 is physically connected and/or attached to the catheter 210. For instance, the sponge 201 may be physically connected and/or attached to the proximal end of the catheter 210 (e.g., such that the sponge physically covers the plurality of holes 202). In at least one example, the sponge 201 may be fitted over an outside circumference of the catheter 210. Thus, the sponge 201 may be of a cylindrical, or substantially cylindrical, shape with a hollow central portion such that the proximal end of the catheter 210 can be inserted within the central portion.

In at least one example, the sponge 201 may be microporous such that fluid (e.g., any fluid to be removed from the body cavity of a patient) can enter and/or pass through the sponge. In at least a further example, the sponge 201 can be an absorbable and/or absorbent sponge. The sponge may be composed of one or more suitable materials, including, for instance, polyurethane, gel/gelatin, and the like.

In use, the fluid removal device 200 can be inserted into the body cavity of a patient using one or more methods for inserting catheters (including, for instance, percutaneous catheters) into patients (e.g., using one or more needles, image guides, guidewires, and/or wire sheets). Fluid can then be extracted from the body cavity into, and through, the sponge 201 and into the lumen of the catheter 210 through the plurality of holes 202. The sponge 201 can therefore provide an absorbable and/or absorbent surface that can draw the fluid from the body cavity through the plurality of holes 202 into the catheter 210. The fluid can then pass through the catheter from the proximal end through the distal end 206 and out of the catheter. Moreover, the fluid can be removed from the body cavity through the tip 204.

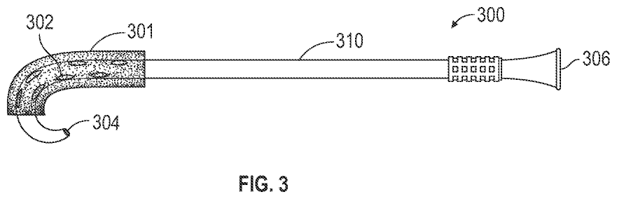

In at least one embodiment, the fluid removal device (e.g., any of the fluid removal devices described herein) comprises a curved catheter (e.g., any of the catheters described herein) and a sponge (e.g., any of the sponges described herein). The catheter is curved or can be adjusted to be curved before or after entering the patient (e.g., inserted straight and adjusted to be curved after being inserted into the patient, for instance, via a suture, to prevent being dislodged). FIG. 3 shows such a fluid removal device 300 in a perspective view. The device 300 comprising a curved catheter 310 and a sponge 301. Similar to catheter 210, the curved catheter 310 comprises a proximal end with a tip 304 and a distal end 306. A plurality of holes 302 are disposed at the proximal end near the tip 304. Further, the curved catheter 310 has an interior lumen in the axial direction, spanning from the proximal end to the distal end. The curved catheter 310 may, in at least one example, have a hollow interior.

In at least one example, the catheter 310 is curved to provide better access to fluid in the body cavity of a patient, thereby enabling better and/or more efficient removal of such fluid. The catheter 310 can therefore be left inside the body cavity for a period of time in order to permit maximum removal and/or drainage of the fluid.

Similar to sponge 201, the sponge 301 is physically connected and/or attached to the curved catheter 310. For instance, the sponge 301 may be physically connected and/or attached to the proximal end of the catheter 310 (e.g., such that the sponge physically covers the plurality of holes 302). In at least one example, the sponge 301 may be fitted over an outside circumference of the catheter 310. Thus, the sponge 301 may be of a cylindrical, or substantially cylindrical, shape with a hollow central portion such that the proximal end of the catheter 210 can be inserted within the central portion.

In at least one example, the sponge 301 may be microporous such that fluid (e.g., any fluid to be removed from the body cavity of a patient) can enter and/or pass through the sponge. In at least a further example, the sponge 301 can be an absorbable and/or absorbent sponge. The sponge may be composed of one or more suitable materials, including, for instance, polyurethane, gel/gelatin, and the like.

As can be seen, the sponge 301 is sufficiently flexible such that it accommodates and/or adapts to the curved portion of the catheter 310. In at least one example, the catheter 310 can be adjusted to be either straight or curved as needed to remove fluid from the body cavity of a patient. For instance, depending on the placement and/or position of the fluid(s) in the body cavity, either a straight or curved catheter may provide better and/or more efficient access and/or removal.

In use, the fluid removal device 300 can be inserted into the body cavity of a patient using one or more methods for inserting catheters (including, for instance, percutaneous catheters) into patients (e.g., using one or more needles, image guides, guidewires, and/or wire sheets). Fluid can then be extracted from the body cavity into, and through, the sponge 301 and into the lumen of the curved catheter 310 through the plurality of holes 302. The sponge 301 can therefore provide an absorbable and/or absorbent surface that can draw the fluid from the body cavity through the plurality of holes 302 into the curved catheter 310. The fluid can then pass through the catheter from the proximal end through the distal end 306 and out of the catheter. Moreover, the fluid can be removed from the body cavity through the tip 304.

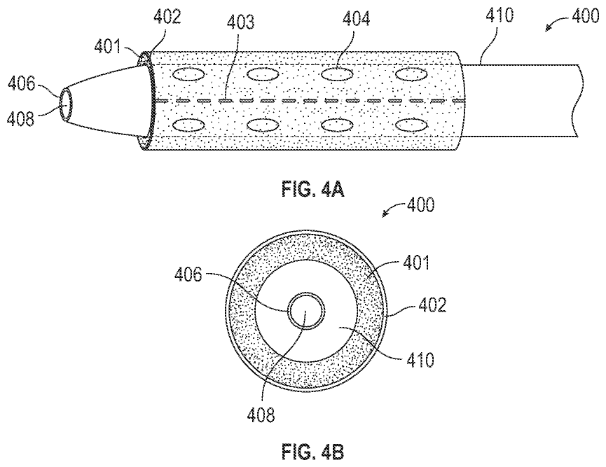

In at least an additional embodiment, a fluid removal device comprises a catheter (e.g., any of the catheters described herein), a sponge (e.g., any of the sponges described herein), and a cover layer that is physically connected and/or attached to an outer surface and/or outer circumference of the sponge. FIG. 4A depicts a perspective view of such a fluid removal device 400. The device comprises a catheter 410 and a sponge 401. Similar to catheters 210 and/or 310, the catheter 410 comprises a proximal end with a tip 406 and a distal end (not shown). A plurality of holes 404 are disposed at the proximal end near the tip 406. Further, the catheter 410 has an interior lumen 408 in the axial direction, spanning from the proximal end to the distal end. The catheter 410 may, in at least one example, have a hollow interior.

FIG. 4B depicts an axial view of the fluid removal device 400. Specifically, the axial view is taken from the proximal end of the device, and more specifically, from the proximal view of the catheter 410. As in FIG. 4A, the proximal end 406 and the interior lumen 408 are shown. The sponge 401 is physically connected and/or attached to the outer surface and/or outer circumference of the catheter 410. The cover layer 402 is physically connected and/or attached to the outer surface and/or outer circumference of the sponge 401.

Similar to sponges 201 and/or 301, the sponge 401 is physically connected and/or attached to the catheter 410. For instance, the sponge 401 may be physically connected and/or attached to the proximal end of the catheter 410 (e.g., such that the sponge physically covers the plurality of holes 402). In at least one example, the sponge 401 may be fitted over an outside circumference of the catheter 410. Thus, the sponge 401 may be of a cylindrical, or substantially cylindrical, shape with a hollow central portion such that the proximal end of the catheter 410 can be inserted within the central portion.

In at least one example, the sponge 401 may be microporous such that fluid (e.g., any fluid to be removed from the body cavity of a patient) can enter and/or pass through the sponge. In at least a further example, the sponge 401 can be an absorbable and/or absorbent sponge. The sponge may be composed of one or more suitable materials, including, for instance, polyurethane, gel/gelatin, and the like.

Additionally, cover layer 402 is disposed, and/or physically connected and/or attached to, an outer surface of the sponge 401. The cover layer may be composed of one or more suitable materials (e.g., cellulose). The cellulose may, in at least one example, be an absorbable and/or absorbent cellulose. In at least one example, the cover layer 402 comprises one or more releasable portions 403 that allow the cover layer 402 to be released from the sponge 401. Such release can happen either before or after insertion of the fluid removal device 400 into the body cavity of the patient. The one or more releasable portions 403 may, in at least one example, comprise a pre-cut portion or line, removal of which causes the cover layer 402 to detach from the sponge 401. For instance, the portion 403 may be pre-cut such that exertion of force on such portion causes the cover layer 402 to detach from the sponge 401. The cover layer 402 can then be removed from the body cavity of the patient (e.g., by a medical professional or clinician treating the patient).

In at least one embodiment, the cover layer 402 constricts and/or restricts the diameter and/or size of the sponge 401, which may be necessary for insertion of the fluid removal device 400 into the body cavity of the patient (e.g., through one or more incisions in the skin and/or tissues of the patient). Removal of the cover layer 402 (e.g., via removal of the one or more releasable portions 403) can occur after the fluid removal device 400 has been sufficiently inserted into the body cavity of the patient. Such removal may cause the sponge 401 to expand to its normal, uncompressed diameter and/or size. Accordingly, various different sizes of sponges can be used in the fluid removal devices described herein, depending on the area of the body cavity surface to be drained.

In use, the fluid removal device 400 can be inserted into the body cavity of a patient using one or more methods for inserting catheters (including, for instance, percutaneous catheters) into patients (e.g., using one or more needles, image guides, guidewires, and/or wire sheets). The cover layer 402 can be removed either before or after insertion of the fluid removal device 400 into the patient. Such removal can be accomplished by, for instance, removal of the one or more releasable portions 403 (e.g., by a medical professional or clinician treating the patient). Fluid can then be extracted from the body cavity into, and through, the sponge 401 and into the lumen 408 of the catheter 410 through the plurality of holes 404. The sponge 401 can therefore provide an absorbable and/or absorbent surface that can draw the fluid from the body cavity through the plurality of holes 404 into the catheter 410. The fluid can then pass through the catheter from the proximal end through the distal end and out of the catheter. Moreover, the fluid can be removed from the body cavity through the tip 408.

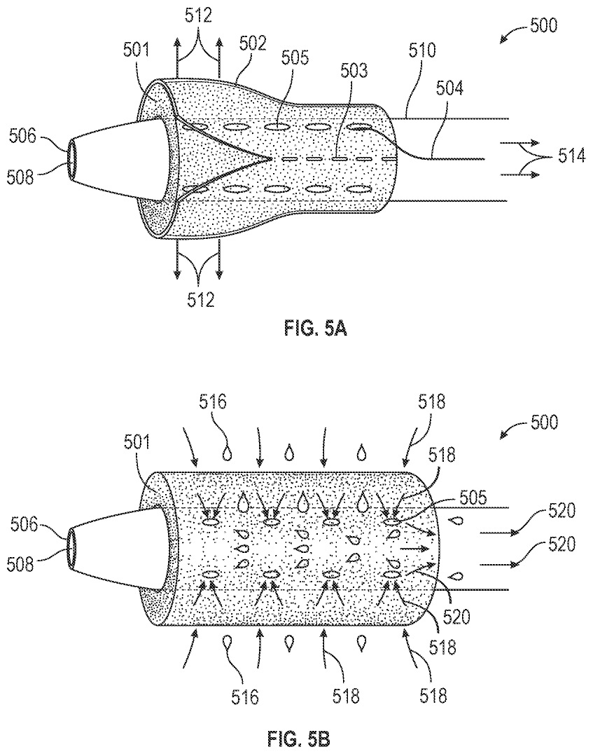

In at least one embodiment, a fluid removal device is disclosed herein that can be used in conjunction with a suction, vacuum and/or negative pressure device and/or system (e.g., a negative pressure (NPT) device and/or system). Such a suction, vacuum and/or negative pressure device and/or system can provide negative pressure and/or sub-atmospheric pressure to assist in extracting fluid from the fluid removal device. In at least one example, any fluid removal device disclosed herein can be used in conjunction with a suction, vacuum and/or negative pressure device and/or system. Turning now to FIGS. 5A-5B, perspective views of a non-limiting example of such a fluid removal device are shown. Fluid removal device 500 comprises a catheter 510 and a sponge 501. Similar to catheters 210, 310, and/or 410, the catheter 510 comprises a proximal end with a tip 506 and a distal end (not shown). A plurality of holes 505 are disposed at the proximal end near the tip 506. Further, the catheter 510 has an interior lumen 508 in the axial direction, spanning from the proximal end to the distal end. The catheter 510 may, in at least one example, have a hollow interior.

Similar to sponges 201, 301, and/or 401, the sponge 501 is physically connected and/or attached to the catheter 510. For instance, the sponge 501 may be physically connected and/or attached to the proximal end of the catheter 510 (e.g., such that the sponge physically covers the plurality of holes 505). In at least one example, the sponge 501 may be fitted over an outside circumference of the catheter 510. Thus, the sponge 501 may be of a cylindrical, or substantially cylindrical, shape with a hollow central portion such that the proximal end of the catheter 510 can be inserted within the central portion.

In at least one example, the sponge 501 may be microporous such that fluid (e.g., any fluid to be removed from the body cavity of a patient) can enter and/or pass through the sponge. In at least a further example, the sponge 501 can be an absorbable and/or absorbent sponge. The sponge may be composed of one or more suitable materials, including, for instance, polyurethane, gel/gelatin, and the like.

Additionally, cover layer 502 is disposed, and/or physically connected and/or attached to, an outer surface of the sponge 501. The cover layer may be composed of one or more suitable materials (e.g., cellulose). The cellulose may, in at least one example, be an absorbable and/or absorbent cellulose. In at least one example, the cover layer 502 comprises one or more releasable portions 503 that allow the cover layer 502 to be released from the sponge 501. Such release can happen either before or after insertion of the fluid removal device 500 into the body cavity of the patient. The one or more releasable portions 503 may, in at least one example, comprise a pre-cut portion or line, removal of which causes the cover layer 502 to detach from the sponge 501. For instance, the portion 503 may be pre-cut such that exertion of force on such portion causes the cover layer 502 to detach from the sponge 501. The cover layer 502 can then be removed from the body cavity of the patient (e.g., by a medical professional or clinician treating the patient.

In at least one embodiment, the cover layer 502 constricts and/or restricts the diameter and/or size of the sponge 501, which may be necessary for insertion of the fluid removal device 500 into the body cavity of the patient (e.g., through one or more incisions in the skin and/or tissues of the patient). Removal of the cover layer 502 (e.g., via removal of the one or more releasable portions 503) can occur after the fluid removal device 500 has been sufficiently inserted into the body cavity of the patient. Such removal may cause the sponge 501 to expand to its normal, uncompressed diameter and/or size. Accordingly, various different sizes of sponges can be used in the fluid removal devices described herein, depending on the area of the body cavity surface to be drained.

In use, the fluid removal device 500 can be inserted into the body cavity of a patient using one or more methods for inserting catheters (including, for instance, percutaneous catheters) into patients (e.g., using one or more needles, image guides, guidewires, and/or wire sheets). The cover layer 502 can be removed either before or after insertion of the fluid removal device 500 into the patient. Such removal can be accomplished by, for instance, removal of the one or more releasable portions 503 (e.g., by a medical professional or clinician treating the patient). FIG. 5A shows the releasable portion 503 in the process of being detached and/or removed. Such removal occurs can occur through exertion of force (e.g., by a medical professional or clinician treating the patient) on suture 504, which can be physically connected and/or attached to releasable portion 503. Thus, exertion of force on suture 504 causes releasable portion 503 to detach and/or release the cover layer 502 from the sponge 501. As mentioned above herein, and as shown in FIG. 5A, removal of the cover layer 502 can cause the sponge 501 to expand outward in direction 512. This can provide additional area for absorption of the fluid to be removed and/or drained.

This fluid can then be extracted from the body cavity into, and through, the sponge 501 and into the lumen 508 of the catheter 510 through the plurality of holes 505. The sponge 501 can therefore provide an absorbable and/or absorbent surface that can draw the fluid from the body cavity through the plurality of holes 505 into the catheter 510. The fluid can then pass through the catheter from the proximal end through the distal end and out of the catheter, along direction 514. Moreover, the fluid can be removed from the body cavity through the tip 506.

FIG. 5B further depicts the movement of fluid into the fluid removal device 500. In this figure, sponge 501 is shown in its fully expanded form. Thus, the cover layer 502, as well as the releasable portion 503 and the suture 504, are not shown. Drops of fluid 516 (of which only two are labeled for ease of reference) move, and/or are drawn, into the sponge 501 along direction 518. The fluid 516 is absorbed by the sponge 501 and the fluid then passes through the plurality of holes 505, and into the lumen 508, along direction 518. The fluid 516 then passes along an axial direction of the lumen 508 to a distal end of the catheter (not shown) along direction 520. This brings the fluid 516 out of the catheter; the fluid can then be collected using one or more collection devices (e.g., collection bags) (not shown).

Movement of the fluid into and/or through a fluid removal device, including, for instance, as depicted in FIGS. 5A-5B, can be achieved, either partially or wholly, through the use of a suction, vacuum and/or negative pressure device and/or system (e.g., a negative pressure therapy (NPT) device and/or system). Such a suction, vacuum and/or negative pressure device and/or system can be physically connected and/or attached to the fluid removal device 500 (e.g., through the distal end of catheter 510). Activation of the negative pressure device and/or system can cause negative and/or sub-atmospheric pressure to be applied to, and/or exerted on, the fluid removal device 500 and, in particular, the catheter 510 (including, for instance, the lumen 508) and the sponge 501. This can cause the fluid to be extracted from the patient's body cavity into the sponge 501 and then into the lumen 508 of the catheter 510 (e.g., through the plurality of holes 505). It should be appreciated that any of the fluid removal devices described herein can also be used in conjunction with such an aforementioned suction, vacuum and/or negative pressure device and/or system. In at least one example, the applied negative and/or sub-atmospheric pressure is in the range of −40 mM Hg to −200 mM Hg; that is, 40 mM Hg below atmospheric pressure to 200 mM Hg below atmospheric pressure, or more preferably −80 mM Hg to −150 mM Hg, or still more preferably −100 mM Hg to −150 mM Hg.

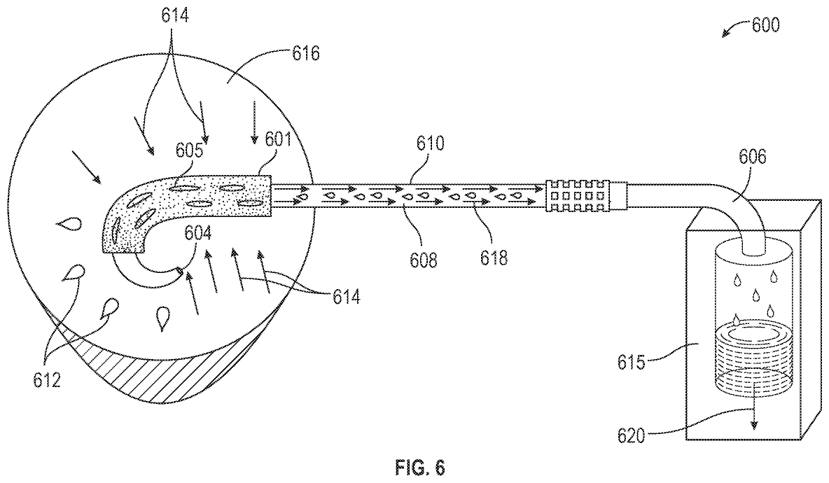

In at least an additional embodiment, a fluid removal system is disclosed herein. Such a fluid removal system incorporates suction, vacuum, and/or negative pressure. As shown in FIG. 6, fluid removal system 600 comprises a catheter 610 and a sponge 601. Similar to catheters 210, 310, 410, and/or 510, the catheter 610 comprises a proximal end with a tip 604 and a distal end 606. Catheter 610 is shown in a curved position, similar to catheter 310. As can be seen, sponge 601 is sufficiently flexible such that it accommodates and/or adapts to the curved portion of the catheter 610. A plurality of holes 605 are disposed at the proximal end near the tip 604. Further, the catheter 610 has an interior lumen 608 in the axial direction, spanning from the proximal end to the distal end 606. The catheter 610 may, in at least one example, have a hollow interior.

Similar to sponges 201, 301, 401, and/or 501, the sponge 601 is physically connected and/or attached to the catheter 610. For instance, the sponge 601 may be physically connected and/or attached to the proximal end of the catheter 610 (e.g., such that the sponge physically covers the plurality of holes 605). In at least one example, the sponge 601 may be fitted over an outside circumference of the catheter 610. Thus, the sponge 601 may be of a cylindrical, or substantially cylindrical, shape with a hollow central portion such that the proximal end of the catheter 610 can be inserted within the central portion.

In at least one example, the sponge 601 may be microporous such that fluid (e.g., any fluid to be removed from the body cavity of a patient) can enter and/or pass through the sponge. In at least a further example, the sponge 601 can be an absorbable and/or absorbent sponge. The sponge may be composed of one or more suitable materials, including, for instance, polyurethane, gel/gelatin, and the like.

The proximal end of the catheter 610, including the sponge 601, is shown within a body cavity 616 of a patient. The body cavity can contain fluid (e.g., abscesses, exudate, harmful material, infections material, and/or infectious agents) to be removed. Insertion of the catheter 610 and the sponge 601 into the body cavity 616 can be achieved using one or more methods for inserting catheters (including, for instance, percutaneous catheters) into patients (e.g., using one or more needles, image guides, guidewires, and/or wire sheets).

The distal end 606 of the catheter is physically connected and/or attached to a suction, vacuum, and/or negative pressure device 615. Such a negative pressure device and/or system may be, for instance, a NPT device and/or system. Briefly, the device 615 operates by exerting continuous or intermittent negative and/or sub-atmospheric pressure (e.g., in the range of −40 mM Hg to −200 mM Hg, or more preferably −80 mM Hg to −150 mM Hg, or still more preferably −100 mM Hg to −150 mM Hg). Such negative and/or sub-atmospheric pressure is applied to, and/or exerted on the catheter 610 (including, for instance, the lumen 608) and the sponge 601. This results in drops of fluid 612 (of which only two are labeled for ease of reference) to be drawn into the sponge 601 (and/or through the tip 604) along direction 614. Such movement removes the fluid 612 from the body cavity 616 of the patient. The fluid 612 is absorbed by the sponge 601 and the fluid then passes through the plurality of holes 605, and into the lumen 608. The fluid 612 moves within the lumen 608 along direction 618, towards the distal end 606 of the catheter 610. Since the distal end 606 is physically connected and/or attached to the negative pressure device 615, the fluid 612 is drawn from the distal end of the catheter into the device 615 along direction 620. The fluid can then be collected (e.g., in one or more collection devices and/or collection bags (not shown)) and/or otherwise disposed of.

The devices, systems, and/or methods for percutaneous-mediated fluid removal described herein can be used to remove fluid from any portion of a subject or patient, not limited to a specific body cavity of the subject or patient. Further, the fluid removal devices, systems, and/or methods described herein provide several advantages over current devices, systems, and methods of fluid removal. Non-limiting examples of such advantages include (1) more complete and/or efficient removal of fluid (e.g., abscesses, exudate, harmful material, infectious material, and/or infections agents) through percutaneous incisions that can be smaller, less disruptive to the patient, and/or less prone to infections or complications than procedures requiring an open body cavity, (2) less invasive and/or minimally invasive compared to current procedures for fluid removal and/or drainage, (3) a larger and/or more effective drainage surface and/or ability compared to current procedures for fluid removal and/or drainage, (4) body cavity diameter reduction, (5) macroscopic and/or microscopic deformation of one or more tissues that can stimulate wound healing, (6) improved healing of internal injuries and/or wounds, including, for instance, wounds that are non-healing or could potentially be non-healing (e.g., skin grafts, diabetes-related ulcers, etc.), (7) improved clearance, control, and/or resolution of infections and/or infectious material (e.g., bacteria, viruses, fungi, parasites, foreign debris, and the like), (8) prevention and/or reduction of additional or secondary infections, (9) enhanced and/or improved blood vessel maturation, (10) improved blood flow that can stimulate healing, (11) improved and/or better-regulated blood flow perfusion, (12) promotion of tissue growth and/or granulation tissue, (13) prevention and/or reduction of inflammation and/or edema, (14) faster patient recovery from internal injuries, wounds, and/or infections, and/or (15) decreased mortality rate for patients suffering from internal injuries, wounds, and/or infections.

Further, the devices, systems, and/or methods for percutaneous-mediated fluid removal described herein can be used in combination and/or conjunction with one or more additional agents administered prior to, in conjunction with, and/or subsequent to application of the fluid removal devices, systems and/or methods of the present disclosure. Non-limiting examples of such additional agents are described below.

Additional Therapeutic, Prophylactic, and/or Diagnostic Agents

In at least one embodiment, the fluid removal devices, systems, and/or methods described herein can be used in combination with one or more additional therapeutic, diagnostic, and/or prophylactic agents to alleviate pain (e.g., pain associated with one or more diseases and/or conditions causing a buildup of fluid such as abscesses, exudate, harmful material, infectious material, and/or infectious agents), facilitate healing, and/or to reduce or inhibit scarring. Non-limiting examples include antimicrobial agents, analgesics, local anesthetics, anti-inflammatory agents, antioxidants, immunosuppressants, anti-allergenic agents, enzyme cofactors, essential nutrients, and growth factors.

In at least one embodiment, the active agents include, for instance, small molecules, biomolecule, peptides, sugar, glycoproteins, polysaccharides, lipids, nucleic acids, and/or combinations thereof. Suitable small molecule active agents include, but are not limited to, organic and organometallic compounds. In at least one instance, the aforementioned small molecule active agent has a molecular weight of less than about 2000 g/mol, more preferably less than about 1500 g/mol, and most preferably less than about 1200 g/mol. The small molecule active agent can be a hydrophilic, hydrophobic, or amphiphilic compound.

In some cases, the active agent is a diagnostic agent imaging or otherwise assessing the patient (e.g., one or more body cavities of the patient that contain fluid to be removed). Exemplary diagnostic agents include paramagnetic molecules, fluorescent compounds, magnetic molecules, and radionuclides, x-ray imaging agents, and contrast media.

In at least an additional embodiment, the fluid removal devices, systems, and/or methods described herein can be used in conjunction with one or more drugs to treat, prevent or diagnose a disease, disorder, or condition (e.g., any disease, disorder, or condition causing a buildup of fluid such as abscesses, exudate, harmful material, infectious material, and/or infectious agents). Non-limiting examples of such drugs include anti-angiogenesis agents, anti-infective agents, anti-inflammatory agents, analgesics, local anesthetics, growth factors, immunosuppressant agents, anti-allergic agents, anti-oxidants, cytokines, and combinations thereof.

In at least a further embodiment, one or more therapeutic, prophylactic, and/or diagnostic agents is administered prior to, in conjunction with, and/or subsequent to application of the fluid removal devices, systems, and/or methods described herein.

In at least a further embodiment, the aforementioned therapeutic, prophylactic and/or diagnostic agents may be administered in a neutral form, or in the form of a pharmaceutically acceptable salt. In at least one example, it may be desirable to prepare a formulation containing a salt of an agent due to one or more of the salt's advantageous physical properties, such as, for example, enhanced stability, a desirable solubility, and/or a desirable dissolution profile.

In at least a further embodiment, pharmaceutically acceptable salts are prepared by reaction of the free acid or base forms of an active agent with a stoichiometric amount of the appropriate base or acid in water or in an organic solvent, or in a mixture of the two; generally, non-aqueous media such as, for example, ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts, as well as salts formed by reaction of the drug with a suitable organic ligand (e.g., quaternary ammonium salts). Lists of suitable salts are found, for example, in Adej are et al., Remington: The Science and Practice of Pharmacy (23rd ed.), Academic Press (2020).

In at least a further embodiment, the fluid removal devices, systems, and/or methods described herein are used and/or applied with one or more local anesthetics. Non-limiting examples of such local anesthetics include tetracaine, lidocaine, amethocaine, proparacaine, lignocaine, and bupivacaine. In at least one example, one or more additional agents, such as, e.g., a hyaluronidase enzyme, is also used to accelerate and/or improve dispersal of the local anesthetic. In some cases, the active agent is an anti-allergic agent such as olopatadine and/or epinastine.

Anti-Infective Agents

In at least one embodiment, the fluid removal devices, systems, and/or methods described herein are used in combination with one or more antimicrobial agents. An antimicrobial agent, at least in the context of the present disclosure, is a substance that inhibits the growth of microbes including, for instance, bacteria, fungi, viruses, and/or parasites. Accordingly, antimicrobial agents include, for example, antiviral agents, antibacterial agents, antiparasitic agents, and anti-fungal agents. Non-limiting examples of antiviral agents include, e.g., ganciclovir and acyclovir. Non-limiting examples of antibiotic agents include, for example, aminoglycosides (e.g., streptomycin, amikacin, gentamicin, and tobramycin), ansamycins (e.g., geldanamycin and herbimycin), carbacephems, carbapenems, cephalosporins, glycopeptides (e.g., vancomycin, teicoplanin, and telavancin), lincosamides, lipopeptides (e.g., daptomycin, macrolides such as azithromycin, clarithromycin, dirithromycin, and erythromycin), monobactams, nitrofurans, penicillins, polypeptides (e.g., bacitracin, colistin, and polymyxin B), quinolones, sulfonamides, and tetracyclines.

Other exemplary antimicrobial agents include, for instance, iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

Anesthetics

In at least one embodiment, the fluid removal devices, systems, and/or methods described herein are administered in combination with one or more local anesthetics. A local anesthetic, at least in the context of the present disclosure, is a substance that causes reversible local anesthesia and has the effect of loss of sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations thereof. In at least one example, one or more anesthetic agents are administered in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of a total composition of a biological and/or pharmaceutical solution and/or formulation. The concentration of local anesthetics can be therapeutically effective, meaning that the concentration is adequate to provide a therapeutic benefit without inflicting harm to the patient.

Anti-Inflammatory Agents

In at least one embodiment, the fluid removal devices, systems, and/or methods described herein are administered in combination with one or more anti-inflammatory agents. Anti-inflammatory agents reduce inflammation and include, for instance, steroidal and non-steroidal drugs. Suitable steroidal active agents include, for example, glucocorticoids, progestins, mineralocorticoids, and corticosteroids. Other non-limiting examples of anti-inflammatory agents include triamcinolone acetonide, fluocinolone acetonide, prednisolone, dexamethasone, loteprednol, fluorometholone, ibuprofen, aspirin, and naproxen. Non-limiting examples of immune-modulating drugs include cyclosporine, tacrolimus, and rapamycin. Non-limiting examples of non-steroidal anti-inflammatory drugs (NSAIDs) include ketorolac, nepafenac, and diclofenac.

In at least an additional embodiment, the anti-inflammatory agents are anti-inflammatory cytokines. Non-limiting examples of such cytokines include IL-10, IL-17, TNF-α, TGF-β, IL-35, and others described herein. Anti-inflammatory cytokines may be cytokines that induce an anti-inflammatory immune environment or suppress an inflammatory immune environment.

Cofactors and Essential Nutrients

In at least one embodiment, the fluid removal devices, systems, and/or methods described herein are administered in combination with one or more enzyme cofactors, and/or one or more essential nutrients. Non-limiting examples of such cofactors include vitamin C, biotin, vitamin E, and vitamin K. Non-limiting examples of such essential nutrients include amino acids, fatty acids, etc.

Polymer Matrices

In at least one embodiment, the fluid removal devices, systems, and/or methods described herein can be administered in combination with one or more therapeutic, prophylactic or diagnostic agents dispersed or encapsulated in a polymeric matrix. The matrix can be formed of non-biodegradable or biodegradable matrices, although biodegradable matrices are preferred. The polymer is selected based on the time required for in vivo stability, e.g., that time required for distribution to the site where delivery is desired, and the time desired for delivery.

Representative synthetic polymers include: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly (ethylene glycol), polyalkylene oxides such as poly(ethylene oxide), polyalkylene terephthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly (butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate.

Examples of preferred non-biodegradable polymers include ethylene vinyl acetate, poly(meth)acrylic acid, polyamides, copolymers and mixtures thereof.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. A device for removing fluid from a body cavity of a patient, the device comprising:
a percutaneous catheter comprising a first end, a second end, and a lumen disposed in an interior of the percutaneous catheter from the first end to the second end, the first end comprising a tip and a plurality of holes fluidly connected to the lumen;
one or more microporous sponges physically connected to the first end of the percutaneous catheter; and
a cover layer covering an outer surface of the one or more microporous sponges,
wherein the percutaneous catheter is configured to curve at the first end,
wherein the one or more microporous sponges are flexible such that the one or more sponges adapt to the curve, and
wherein the cover layer comprises one or more releasable portions that are configured to allow the cover layer to be removed after insertion of the percutaneous catheter and the one or more microporous sponges into the body cavity of the patient.

2. The device of claim 1, wherein at least one sponge of the one or more microporous sponges comprises a hollow center, and wherein the hollow center fits around an outer circumference of the first end of the percutaneous catheter.

3. The device of claim 1, wherein at least one sponge of the one or more microporous sponges physically covers the plurality of holes.

4. The device of claim 1, wherein the one or more microporous sponges are composed of polyurethane and/or gelatin.

5. The device of claim 1, wherein the one or more releasable portions are physically connected to one or more sutures such that exertion of force on the one or more sutures causes the cover layer to be removed.

6. The device of claim 1, wherein the cover layer is composed of absorbable cellulose.

7. The device of claim 1, wherein the cover layer constricts a maximum diameter of the one or more microporous sponges, and wherein removal of the cover layer results in the one or more microporous sponges expanding to the maximum diameter.

8. The device of claim 1, wherein the second end of the percutaneous catheter is physically connected to an apparatus that provides suction through the lumen of the percutaneous catheter.

9. A system for removing fluid from a body cavity of a patient, the system comprising:
a fluid removal device comprising a percutaneous catheter and a sponge attached around an outer circumference of the percutaneous catheter at an end of the percutaneous catheter inserted into the body cavity of the patient; and
an apparatus attached to the fluid removal device,
wherein the percutaneous catheter comprises a lumen that is fluidly connected to the sponge,
wherein the apparatus applies continuous or intermittent negative pressure into both the lumen and the sponge when the fluid removal device is inserted into the body cavity of the patient.

10. The system of claim 9, wherein the sponge is absorbable such that the sponge absorbs the fluid from the body cavity of the patient and passes the fluid into the lumen of the percutaneous catheter.

11. The system of claim 9, further comprising one or more collection bags for collecting the fluid from the body cavity of the patient.

12. The system of claim 9, wherein the negative pressure is in a range from −100 mm Hg to −150 mm Hg.

13. The system of claim 9, wherein the fluid removal device further comprises an outer layer of absorbable cellulose, the outer layer attached to an outer surface of the sponge.

14. The system of claim 13, wherein the outer layer further comprises a pre-cut section attached to a suture such that exertion of force in the suture removes the pre-cut section and releases the outer layer from the outer surface of the sponge.

15. A method for removing fluid from a body cavity of a patient, the method comprising:
inserting a fluid removal device into the body cavity of the patient, the fluid removal device comprising:
a percutaneous catheter comprising a lumen, and
a microporous sponge attached around an outer circumference of the percutaneous catheter, the microporous sponge fluidly connected to the lumen;
applying negative pressure to the fluid removal device via a suction apparatus;
conducting the fluid to be removed from the body cavity of the patient into the microporous sponge and into the lumen, thereby removing the fluid from the body cavity of the patient.

16. The method of claim 15, wherein the percutaneous catheter comprises a plurality of holes fluidly connecting the lumen with the microporous sponge, and wherein the microporous sponge is in a shape of a hollow cylinder.

17. The method of claim 16, wherein the fluid to be removed from the body cavity of the patient is absorbed into the sponge and then passed from the sponge into the lumen via the plurality of holes.

18. The method of claim 15, wherein the applying the negative pressure further comprises:
activating the suction apparatus to exert the negative pressure into the lumen and the sponge.

19. The method of claim 15, wherein the negative pressure is in a range from −100 mM Hg to −150 mM Hg.

20. The method of claim 15, further comprising:
collecting the fluid removed from the body cavity of the patient in one or more collection devices.
